(19) United States
(12) Patent Application Publication
(10) Pub. No.: US 2020/0268656 A1
(43) Pub. Date: Aug. 27, 2020

(54) Nebulized Ethanol for Internal Disinfecting and Improvement

(71) Applicants: Darren Rubin, Largo, FL (US); Howard Rubin, Land O Lakes, FL (US)

(72) Inventors: Darren Rubin, Largo, FL (US); Howard Rubin, Land O Lakes, FL (US)

(21) Appl. No.: 16/874,455

(22) Filed: May 14, 2020

(51) Int. Cl. A61K 9/00; A61K 31/045; A61K 9/08

(52) U.S. Cl. CPC A61K 9/0078; A61K 45/06; A61K 9/08; A61K 31/045

(57) ABSTRACT

Methods of the present disclosure utilize alcohol (e.g., ethanol) as an active ingredient or agent for disinfecting, washing, and/or treating infection in the upper and/or lower airways. Methods include aerosol inhalation, lung spraying, and/or lung lavage using ethanol-containing solutions. Such methods can be applied to complicated pneumonia and respiratory pandemics, including the COVID-19 pandemic caused by severe acute respiratory syndrome coronavirus 2 (SARS-CoV-2).

### BACKGROUND

Treatment protocols for viral infections are often specific to the virus strain, with little efficacy crossover between virus strains. In some examples, available treatments for one type of virus strain may be ineffective against a mutation of that virus strain. As a result, there is often a significant lag between discovery of an infectious virus strain and an effective treatment protocol being available. Sometimes the lag can be on the order of years. For virus strains that pose significant health risks, such as severe acute respiratory syndrome coronavirus 2 (SARS-CoV-2), which causes COVID-19 disease, the lack of treatment and prevention can lead to global pandemics. Technological advancements of viral treatments may also be held back by the speed at which a virus mutates. For example, due to a lack of financial return on investment, research and development of a treatment for a particular virus strain may be scuttled if the virus strain mutates to a new, less dangerous strain before the treatment is fully developed and evaluated. As such, there remains a desire for a more comprehensive treatment of viral infections.

### DETAILED DESCRIPTION

This disclosure includes methods for treatment of respiratory viral infections using alcohol (e.g., ethanol) as an active ingredient or agent for disinfecting, washing, and/or treating infection in the upper and/or lower airways. The various methods described herein include aerosol inhalation, lung spraying, and/or lung lavage using ethanol-containing solutions. Particularly relevant to the current COVID-19 pandemic, the treatments developed according to this disclosure may be particularly helpful to mitigate effects of the SARS-CoV-2 and the COVID-19 disease, which may result in many lives being saved.

Alcohol in the healthcare setting generally includes ethyl alcohol (ethanol) or isopropyl alcohol, yet many other alcohols exist. Ethanol lock is sometimes used for infusion catheter lines, e.g., central venous catheter lines. The Centers for Disease Control (CDC) note that alcohol can be used as a chemical disinfectant, but point out that alcohols have generally underrated germicidal characteristics. It is believed that no liquid chemical sterilant or high-level disinfectant containing alcohol as a main active ingredient has been cleared by the United States Food and Drug Administration (FDA). The CDC is aware of hospital infection outbreaks that have occurred when alcohol was used to disinfect medical instruments, instead of more effective disinfectants. Alcohol does not destroy bacterial spores. It is clear to see why alcohol is not the best disinfectant in the healthcare setting.

Yet, the methods of the present disclosure utilize alcohol as an active ingredient or agent for disinfecting and/or treating infection in the upper and/or lower airways. Ethanol may be used in some embodiments. Additionally or alternatively, another alcohol (e.g., isopropyl alcohol) and/or a different virucidal and/or bactericidal agent may be used. In many embodiments of these inventive methods, an alcohol product (e.g., a liquid pharmaceutical drug composition or formulation containing alcohol), or a dilution or solution thereof, is used. For example, a liquid pharmaceutical drug product and/or formulation that includes ethanol (e.g., dehydrated alcohol or dehydrated alcohol injection), or a dilution or solution thereof, is used. A liquid ethanol product or solution may be applied to the airways as described herein for disinfection, reduction of viral load, treatment, improving lung function, improving oxygen saturation, reducing the need for ventilation (e.g., mechanical ventilation), or a combination thereof. For example, an ethanol product may be applied to airways of patients having coronavirus or have been exposed to coronavirus.

The methods described herein may also be counterintuitive because alcohol intake is a well-known risk factor for developing lung infections; heavy alcohol consumption impairs lung defenses. Alcohol consumption is believed associated with prolonged cough and bronchitis. Moreover, alcohol itself is an ablative agent, when injected into tissues and nerves (e.g., alcohol septal ablation or therapeutic neurolysis).

The respiratory tract includes the upper respiratory system; the upper airways (e.g., the oral cavity, nasal cavity, nasopharynx, oropharynx, laryngopharynx, larynx, and top of the trachea). The lower airways or lower respiratory tract includes the trachea, the bronchi; upper and middle and lower lobes of the lung; bronchioles and alveoli. There may be some overlap of the trachea in these upper and lower airways definitions.

The described methods may be applied to people having SARS-CoV-2, or other viral and/or bacterial exposure, other than coronavirus or coronavirus exposure, or comorbid with coronavirus or coronavirus exposure. For example, the patient may be exposed to SARS-CoV-2 and/or have COVID-19 disease comorbid with an additional or different viral or bacterial infection. Additional or different viral or bacterial infections and infection combinations are within the scope of this disclosure. For example, infections and combinations of infections that include any of a coronavirus, an influenza virus, and/or bacterial pneumonia (e.g., Streptococcus pneumoniae), etc. The described methods may be applied to any primary and/or secondary infection that negatively impacts the airways, and particularly, the lungs.

For example, SARS-CoV-2 is often spread by coughing or sneezing, which releases tiny, virus laden droplets in the air that are inhaled by others in proximity. The infection may begin in the upper airways, such as mucous membranes in the nasal passages and in the back of the throat; then spreads down into the lower airways. The virus attacks the respiratory mucosa. The ability to disinfect these respiratory mucosa and/or prevent (further) spread of the infectious agent (e.g., SARS-CoV-2) may be the difference between life and death in some cases, and may greatly reduce the transmission to others.

The delivery of alcohol (e.g., ethanol) and/or other active ingredients or agents of the described methods include delivery to the airways by spraying, dispensing, dripping, aerosolization, nebulization, delivery that includes application of positive pressure with a positive pressure device (e.g., intermittent positive pressure breathing device), and/or delivery that includes manual and/or mechanical ventilation, or any combination thereof. A positive pressure device can help push the alcohol (e.g., ethanol) further into the lungs. Positive pressure may facilitate this transport with a therapeutic gas, such as oxygen, to the alveolar region.

In some embodiments, an incentive device and/or inhalation resistance device is used as a nebulizer accessory, or a nebulizer with resistance settings (e.g., U.S. Pat. No. 9,757,528), may help enhance aerosol entrainment and delivery to deeper reaches of the lung, and provide pulmonary physiotherapy. Some devices may also provide positive expiratory pressure and/or airway vibrations to help dislodge and/or remove mucous secretions in the airways, but also to help the patient exhale more of their carbon dioxide. An incentive spirometer with positive expiratory pressure features for use with a nebulizer can be ideal in some embodiments.

The alcohol (e.g., ethanol) may be delivered with one or more other active ingredients or agents, such as one or more of each of a bronchodilator (e.g., beta-adrenergic bronchodilators like salbutamol [albuterol], pirbuterol, and terbutaline, or possibly anticholinergic bronchodilators, such as ipratropium bromide), a surfactant (e.g., calfactant), an anti-viral drug (e.g., remdesivir), an antibacterial drug (e.g., azithromycin), an anti-inflammatory drug (e.g., hydroxychloroquine, fluticasone or other steroid or non-steroidal drug), an anti-clotting drug or anticoagulant therapy (e.g., heparin, tissue plasminogen activator), a cytokine (e.g., interferon beta), a cytokine inhibitor, an interleukin, an interleukin inhibitor, other drugs, or any combination thereof. These examples are not meant to be limiting. In other embodiments, the above active ingredients may be delivered separately or sequentially from the alcohol delivery. Some active ingredients can be delivered directly to the lungs, while other may be delivered by other routes, such as oral or intravenous routes. For example, remdesivir (e.g., 200 mg on day one and 100 mg for several days to over a week) may be delivered intravenously to severe COVID-19 patients.

Some infections and diseases, such as COVID-19, are associated with blood clots. Blood clots can form in appendages, which require amputation. Blood clots can also form in the small vessels of the lungs, leading to pulmonary embolism and respiratory failure. Alcohol (e.g., ethanol) has some blood thinning properties and is suspected of reducing clotting processes. It is believed that the methods of this disclosure, such as of delivering alcohol (e.g., ethanol) into the lungs, may help break up blood clot formation in the lungs and/or help prevent blood clotting in the lungs. Furthermore, additional anti-clotting treatments and/or drugs, delivered systemically and/or delivered into the lungs with these methods may greatly help these patients.

A bronchodilator (e.g., 2.5 to 5 mg of salbutamol) may be delivered sequentially or simultaneously with ethanol to counter some of ethanol's suspected pulmonary vasoconstricting properties. In some instances, ethanol may stimulate the bronchodilation. Ingestion of alcohol is known to treat and to cause asthmatic episodes. Inhalation of ethanol has been known to cause irritation and even bronchospasm, although may also relax bronchial smooth muscle. So ethanol has opposing effects, some of which may involve its metabolites (e.g., acetaldehyde).

For COVID-19, a disease marked by alveolar inflammation and low oxygen saturation; treatment to the deepest parts of the lungs may be desirable. The alveoli are where gas exchange takes place in the lungs. The SARS-CoV-2 virus tends to clog these alveoli, such as with virus laden secretions, dead lung cells, and possibly with immune cells as well. The presence of these substances in the alveoli can interfere with the diffusion of oxygen, which epithelial cells lining the alveoli depend. Anoxic epithelium can become more permeable to fluids. A froth may develop from labored breathing which further blocks oxygenation of the alveolar epithelium. In some examples, a secondary effect of these methods involving alcohol is to separate or disrupt this froth, if present. However, it is noted that ethanol can be suspected of having some pulmonary vasoconstricting properties and in some situations, can induce pulmonary edema; even though ethanol has been tried for treating pulmonary edema in the past decades ago. Ethanol is believed to have potential for stabilizing lung surfactant.

It may be desirable to have the alcohol (e.g., ethanol) delivered along with oxygen gas for inhalation. In other embodiments, the alcohol (e.g., ethanol) is delivered with compressed air or other medical gas. For example, a humidifier or humidifier jar can administer ethanol vapor while connected to an oxygen gas supply or flowmeter. Generally, at least 10% ethanol should be placed in the humidifier bottle, and 70% up to absolute ethanol or dehydrated alcohol (e.g., 95% ethanol or more) may be used. It is generally believed that lower concentrations of 10% to 30% are more tolerable with less irritation. Alcohol can irritate the bronchial mucosa. However, higher concentrations (e.g., above 30%, such as 50%, 70% or 95%) may be more denaturing and virucidal. For example, 70% ethanol in water or saline can be placed in a humidifier jar with an oxygen gas flow rate of 2 liters per minute up to 16 liters per minute. In other examples, high flow oxygen systems (up to 60 liters per minute of oxygen) can be used. It is generally recommended that the concentration of ethanol be reduced as the oxygen flow rate increases and/or as the treatment time increases. Treatment times can vary from minutes to over an hour. In some embodiments, warm humidified alcohol solution can be administered and/or a warming humidifier and/or a humidifier bottle warmer can be used when delivering humidified alcohol solution for inhalation, such as with medical gas (e.g., oxygen gas) delivery. Warming inhalation droplets can be more comfortable and prevent drying of the airways, but may also improve (e.g., reduce) particle size for inhalation.

In other examples, ethanol can be delivered by a jet nebulizer while connected to oxygen gas, which operates the jet. In these implementations, oxygen saturation can improve immensely when the liquid ethanol or ethanol solution is inhaled via mouthpiece or by face mask/aerosol mask or by nasal cannula. Compressed air or other medical gas may operate the jet nebulizer. There exist small volume and large volume nebulizers. Alcohol (e.g., ethanol) can also be delivered via an anesthesia mask. In addition to disinfecting the lungs, inhaled ethanol may also break up phlegm and improve expectorate as another advantage. There also exist nebulizer warmers/heaters that can be used in some embodiments to heat the aerosols.

Nebulizers and humidifiers can be used in mechanical ventilation circuits as well.

Hydrogen gas is another medical gas that may also be desired for delivery to the lungs, with or without concomitant oxygen gas therapy. Hydrogen gas can also be delivered via a jet nebulizer or mask or nasal cannula, for example. It is believed that hydrogen gas may reduce some of the hyperoxic lung injury; damage that concentrated oxygen causes to lungs, especially of critically ill patients receiving oxygen gas for extended periods of time, which can lead to respiratory failure. Hydrogen gas has antioxidant and anti-inflammatory effects. Hydrogen gas is believed to protect the lungs at least by regulation of protective proteins, such as by induction and/or activation of heme-oxygenase-1 enzyme. Importantly, hydrogen gas therapy is believed to eliminate toxic reactive oxygen species, such as cytotoxic oxygen radicals; which may accompany inflammation. Reactive oxygen species may constrict airway smooth muscle. These methods utilize hydrogen gas to counter the reactive oxygen species generated from infection-related inflammation in the lungs and/or from oxygen therapy itself. Hydrogen gas may also reduce acute lung injury caused by mechanical ventilation.

Therapeutic gases, such as oxygen and/or hydrogen can also or alternatively be delivered to the airways and/or lungs via liquid solution, such as when the solution is hyperoxygenated and/or hyperhydrogenated. In such embodiments, the liquid solution can contain the alcohol (e.g., ethanol) as well.

Note that ethanol is flammable. Ethanol at 30% is believed flammable at room air, while ethanol at 25% is believed flammable in oxygen. At lower concentrations, ethanol loses its flammability. Caution in storing and using alcohol (e.g., ethanol) is advised, such as keeping away from sources of heat or flame.

In some examples, at least 0.1 mL of alcohol (e.g., ethanol) product or solution may be administered to the upper airways, such as intranasally; e.g., with a nasal spray pump or MDI. For nebulization, an at least 1 mL of ethanol product or solution may be delivered. In some examples, 3 mL or more of ethanol product or solution may be nebulized. An ultrasonic nebulizer can also be used instead of a jet nebulizer, in some examples.

Ethanol inhalation or delivery to airways should be done under the supervision and/or advice of a healthcare practitioner. Generally, nebulizers and other aerosol delivery devices require a prescription. Some nebulizers may have a filter accessory to reduce exposure, (e.g., occupational exposure) to those present in the room.

Other aerosolization devices may also be used. For example, a metered dose intranasal inhaler may be used to deliver alcohol (e.g., ethanol) product or solution droplets to the nasal cavity for sterilization or disinfection of viral load there. Such methods may also reduce the amount of live virus spread to others.

It is to be understood that in many embodiments, the alcohol (e.g., ethanol) is not an excipient ingredient, but rather an active ingredient of the liquid when utilized in many of these methods. Example excipient ingredients may include glycerin, salts like sodium chloride and potassium bicarbonate, detergents and/or chelators such as EDTA, certain polymers, pH adjustors (e.g., acids or bases), liposomes, preservatives, solvents or water. These classes of excipients and/or excipient examples are not meant to be limiting. Pharmaceutically acceptable excipients are preferred.

In some embodiments of these methods, a pulmonary catheter may be used to dispense ethanol product or solution to the airways or lungs. If inhalation of aerosol (e.g., via a mouthpiece) does not reach the alveolar region; dispensing to or near the alveolar region of the lungs can be possible with such an intrapulmonary catheter. This procedure generally may involve bronchoscopy. A fiber-optic bronchoscope device would be desirable in this case, and visualization and/or imaging may be helpful. In some embodiments of these methods, the catheter dispenses or sprays the ethanol containing liquid. In other embodiments to these methods, the catheter includes a tiny intrapulmonary aerosol generation device associated with the catheter and is placed down into the lungs to deliver aerosol directly to the desired regions of the lungs. Some embodiments of these methods rely on performing an endoscopy, including rhinoscopy, laryngoscopy, bronchoscopy, or a combination thereof, to visualize the upper and/or lower airways and/or to apply the alcohol (e.g., ethanol) to the upper and/or lower airways.

Dilution of the liquid ethanol product or solution, such as with water, saline, and/or other drug solution may be generally desirable. A final ethanol product or solution dilution of 30%, 40%, 50%, 60% or 70%, or even above 70% (e.g., 80%) may be preferred. It is believed that pure ethanol is less effective than diluted ethanol because water aids in the denaturation of proteins, which is believed the mode of action for alcohol's disinfectant activity. However, the lungs contain fluid too, at least on its mucosa surface, which can dilute alcohol further. Too much dilution of alcohol may reduce or eliminate alcohol's virucidal/bactericidal activity. It is believed that a final ethanol concentration of 60% to 80% has potent virucidal activity. Yet, too strong a concentration of ethanol could destroy lung tissue. Injection of concentrated ethanol is ablative to tumors, nerves, and other tissues. So finding the right balance between having some virucidal and/or bactericidal activity, and minimizing tissue damage, may be essential with the methods described herein. In some examples, preparation of treatment protocols according to these methods may be further refined and/or customized based on an individual patient's needs. In other embodiments, the final ethanol product or solution dilution may be below 30% alcohol.

Exposure time for the virucidal and/or bactericidal agent, such as alcohol, is another factor. A minimum exposure time may be needed, which can be on the order of a few seconds (e.g., at or around 15 seconds) or on the order of several minutes (e.g., more than 5 minutes). These are nonlimiting examples. The proper exposure time to the alcohol and/or other virucidal and/or bactericidal agent in the airways may depend on the patient, his or her condition, level of mucous secretions, etc. Exposure time may have to be adapted during the time of each procedure until more standardized protocols are developed for these methods.

In some examples, a bronchoalveolar lavage (e.g., lung lavage or lung washing) procedure may be used to provide the ethanol product or solution to the infected patient's lung to wash out the lung and to disinfect and reduce a viral load in the patient's lungs of an infected patient. In some examples, whole lung lavage is desirable. Candidates for this procedure may have experienced a decline in lung function, a decline in partial pressure of oxygen (e.g., to less than 70 mmHg), a decline in diffusing capacity of the lungs for carbon monoxide, a decline in forced vital capacity, a decline in peripheral capillary oxygen saturation, or a combination thereof. Worsening of lung function of candidates for this procedure may also be determined based on imaging of the lungs and/or chest, e.g., a chest X-ray or a computed tomography.

During the lung lavage procedure, generally a bronchoscope is passed through the mouth, trachea, or nose and into an appropriate airway in the lungs. The patient is typically anesthetized under general anesthesia during the procedure to mitigate discomfort and to improve the likelihood that the patient will remain still during the procedure. In some examples, one lung is washed at a time, although both lungs can often be lavaged sequentially in the same treatment session, or in different sessions. It may be advisable to perform the lavage on the most severely affected lung first. In other examples, both lungs may be lavaged simultaneously, although this is less common as it typically requires extracorporeal membrane oxygenation (an artificial lung for heart-lung bypass support). Extracorporeal membrane oxygenation may be required for more severe patients as well.

In this disclosure, an alcohol (e.g., ethanol) product or solution is used for, or to make, at least some portion of the lung lavage wash solution in this procedure and is preferably in normal saline. In some examples, the alcohol (e.g., ethanol) product or solution may be warmed to around 37 degrees Celsius (e.g., body temperature). In some examples, only one or a few lung volumes of lung wash solution contains ethanol. In other examples, most or all of the lung wash solution volume contains the ethanol. The alcohol (e.g., ethanol) comprising the lung wash solution is at a concentration of at least 5% volume by volume of solution, and preferably above 10% volume by volume of solution. Ethanol is also sometimes measured as weight per volume of solution. In some examples, the total lung wash solution volume used is 20 to 30 liters, and up to 50 liters in extreme cases. Therefore, in most examples, at least a half-liter of ethanol-containing lung wash solution is used. In some examples, one liter of ethanol-containing lung wash solution is used. In still further examples, two or more liters of ethanol-containing lung wash solution is used. It is useful to select an alcohol and salt concentration for the aqueous wash solution or tincture wash solution that does not cause the alcohol to become appreciably immiscible in water. Mixing of the alcohol-containing wash solution may be appropriate in some examples. Because of potential salting out, alcohol may not be intuitive to use with saline.

Generally, the wash solution is allowed to flow by gravity (or hydrostatic pressure) into the lung. In some examples, the lung or lungs are filled with wash solution to functional residual capacity, with repeated cycles of instillation and drainage. The initial instillation in some examples is half the functional residual capacity. In other examples, incremental tidal volumes of a half-liter to one liter, or simply one-liter aliquots, of wash solution are instilled. These examples are not meant to be limiting. As solution and effluent is brought out (e.g., suctioned out) of the lungs, initial volumes may be opaquer and less clear, as they contain more mucous and/or deposits. During the procedure, physiotherapy/physical therapy of the thorax and/or chest, manually or with a shaking device or vest, helps agitate the lungs and lung wash to remove more deposits. So chest percussion may be used at both maximal wash volume and during draining, for each cycle. For example, multiple cycles of lung instillation (with of a tidal volume of a half-liter to full liter of warmed wash solution), chest percussion therapy (for a couple minutes; 2 to 5 minutes), and removal of fluid from the lung (by active suction and or drainage) is performed repeatedly; generally until the lavage effluent is clear. Lung ventilation is used during this procedure, as is patient monitoring, such as of vital signs. Pulse oximetry and even serial readings of arterial blood gas levels are checked regularly during this procedure.

A lung lavage wash solution could be hyperoxygenated and/or hyperhydrogenated before use to enhance treatment with these methods. For instance, hyperoxygenated solution may improve oxygen supply and reduce hypoxemia during the procedure. Such solutions can be aerated with oxygen until pressure in its container or bag reaches 1000 mmHg, then warmed to 37 degrees Celsius, prior to lavage use.

In some examples, application of an alcohol (e.g., ethanol) product or solution is administered to the lungs and/or airways prior to, during, and/or after a lung lavage. Suction with a suction device into a specimen collection trap, or collection with a syringe, can remove viral containing fluids and secretions. Applying of the ethanol product or solution to the lungs and/or airways after the lung lavage can help ensure disinfection of the lungs and/or airways and viral load reduction.

Specimen collection from the lung lavage can be used for testing lung fluid and/or lung secretions for virus quantitatively and/or qualitatively, before and/or after the lung lavage procedure; and in most cases, to note a reduction of viral load. In other examples, bacteria and/or fungus in the lung fluid and/or lung secretions may also or alternatively be tested quantitatively and/or qualitatively. Identification of the type of virus and/or bacteria and/or fungus may be beneficial. In some embodiments, identification of the contagion is achieved by antibody binding assays and/or genetic sequencing.

Alcohol-containing lavage wash solutions may also be used in other embodiments, including for pharyngeal lavage and/or nasal lavage (e.g., nasal irrigation). These other embodiments may also disinfect and/or clean out these regions of mucous and contagion. Fluids and/or secretions contained by these other lavages or washes may also be collected and tested for contagion quantitatively and/or qualitatively. In its simplest other embodiment, alcohol-containing wash solution is delivered by a neti pot.

One of the goals of this disclosure is to prevent or treat complicated pneumonia, which otherwise may require lung(s) to be drained or diseased lung tissue to be removed in what is called lung scraping. In some embodiments, lungs may be drained or scraped along with these methods.

In some examples, the methods disclosed herein could be applied to treat soldiers and military personnel that are thought to have been exposed to infectious agents or have active infections.

The methods disclosed herein could be applied to treat veterinary patients or animals that are thought to have been exposed to infectious agents or have active infections. It is believed that zoo animals, such as tigers, had COVID-19 infection. These methods could be applied to pets such as cats and dogs. These methods could be applied to chimpanzees and gorillas. These methods could be applied to endangered species. These methods could be used to treat horses. These methods could be used to treat cattle, swine, and other livestock that comprise our nation's food supply. Although these methods are primarily suited for mammals, the methods could be adapted for treating poultry and other birds. Avian influenza and other future animal epidemics could endanger the world's food supply, and methods for treating such or reducing its spread could be invaluable.

In some examples, a monohydric alcohol may be used.

In some examples, a dihydric or polyhydric alcohol may be used.

In some examples, a non-branched alcohol may be used.

In some examples, a branched alcohol may be used.

In some examples, a ringed alcohol or aromatic alcohol, such as phenol or benzyl alcohol, may be used.

In some examples, two or more alcohol (hydroxyl) functional groups are present, and includes diols and triols for the alcohol.

In some examples, an alcohol derivative or conjugate (e.g., ethyl acetate or butyl acetate) may be used instead or in addition to the alcohol. Alcohol derivatives or conjugates can include esters, ethers, carboxylic acids, amides, amines, ketones, other organic molecules, or combinations thereof.

Blood alcohol levels should be monitored to ensure that significant absorption via the inhalation route is not taking place, so as to avoid potential for alcohol poisoning.

The above examples are not meant to be limiting.

In some embodiments, the alcohol or alcohol product (e.g., a liquid pharmaceutical drug composition or formulation containing alcohol), or a dilution or solution thereof, is comprised of fermented alcohol. For example, ethanol is often made from grain alcohol fermentative processes.

In other embodiments, the alcohol or alcohol product (e.g., a liquid pharmaceutical drug composition or formulation containing alcohol), or a dilution or solution thereof, is comprised of synthesized alcohol. In other embodiments, the alcohol or alcohol product (e.g., a liquid pharmaceutical drug composition or formulation containing alcohol), or a dilution or solution thereof, is comprised of fermented alcohol and synthesized alcohol. For example, the alcohol or alcohol product (e.g., a liquid pharmaceutical drug composition or formulation containing alcohol), or a dilution or solution thereof, is comprised of fermented and/or synthesized ethanol. Chemical synthesis of ethanol can involve the acid-catalyzed hydration of ethylene. In these examples, distillation and dehydration of alcohol may be used to increase the purity of the alcohol. Up to and over 99% pure ethanol can be achievable for the alcohol in some embodiments.

The disclosed methods can be combined with other disinfecting and treatment methods, such as using virucidal and/or bactericidal wavelengths of light shone inside the lungs from a source of light (e.g., via fiber optic(s) and/or associated with a modified bronchoscope) placed down into the lungs.

### Examples

Example 1 is a method, comprising: applying an at least one alcohol as a liquid, solution, or pharmaceutical drug composition to upper and/or lower airways of a person or animal exposed to an infectious agent.

In Example 2, the subject matter of Example 1 additionally or alternatively includes, wherein the upper and/or lower airways of the person or animal exposed to the infectious agent are disinfected in response to application of the at least one alcohol to the upper and/or lower airways of the person or animal.

In Example 3, the subject matter of any of Examples 1-2 additionally or alternatively includes, wherein a viral load in the upper and/or lower airways of the person or animal exposed to the infectious agent is reduced in response to application of the at least one alcohol to the upper and/or lower airways of the person or animal.

In Example 4, the subject matter of any of Examples 1-3 additionally or alternatively includes, treating the person or animal exposed to the infectious agent via application of the at least one alcohol to the upper and/or lower airways of the person or animal.

In Example 5, the subject matter of any of Examples 1-4 additionally or alternatively includes, reducing or eliminating blood clots of the person or animal exposed to the infectious agent in response to application of the at least one alcohol to the upper and/or lower airways of the person or animal.

In Example 6, the subject matter of any of Examples 1-5 additionally or alternatively includes, improving respiration of the person or animal exposed to the infectious agent via application of the at least one alcohol to the upper and/or lower airways of the person or animal.

In Example 7, the subject matter of any of Examples 1-6 additionally or alternatively includes, improving oxygen saturation of the person or animal exposed to the infectious agent via application of the at least one alcohol to the upper and/or lower airways of the person or animal.

In Example 8, the subject matter of any of Examples 1-7 additionally or alternatively includes, preventing a need for mechanical ventilation of the person or animal exposed to the infectious agent via application of the at least one alcohol to the upper and/or lower airways of the person or animal.

In Example 9, the subject matter of any of Examples 1-8 additionally or alternatively includes, reducing a length of time the person or animal exposed to the infectious agent needs mechanical ventilation in response to application of the at least one alcohol to the upper and/or lower airways of the person or animal.

In Example 10, the subject matter of any of Examples 1-9 additionally or alternatively includes, wherein the at least one alcohol includes ethanol.

In Example 11, the subject matter of any of Examples 1-10 additionally or alternatively includes, wherein the at least one alcohol includes propanol or isopropyl alcohol.

In Example 12, the subject matter of any of Examples 1-11 additionally or alternatively includes, wherein the at least one alcohol includes phenol.

In Example 13, the subject matter of any of Examples 1-12 additionally or alternatively includes, wherein an at least one alcohol derivative or conjugate is used instead of or in addition to said at least one alcohol.

In Example 14, the subject matter of any of Examples 1-13 additionally or alternatively includes, nebulizing or aerosolizing the at least one alcohol, or humidifying therapeutic gas with the at least one alcohol, for application to the upper and/or lower airways of the person or animal.

In Example 15, the subject matter of any of Examples 1-14 additionally or alternatively includes, spraying, dispensing, or dripping the at least one alcohol for application of the at least one alcohol to the upper and/or lower airways of the person or animal.

In Example 16, the subject matter of any of Examples 1-15 additionally or alternatively includes, performing an endoscopy, including rhinoscopy, laryngoscopy, bronchoscopy, or a combination thereof, to apply the at least one alcohol to the upper and/or lower airways of the person or animal.

In Example 17, the subject matter of any of Examples 1-16 additionally or alternatively includes, performing a lavage, a bronchoalveolar lavage, or a whole lung lavage to apply the at least one alcohol to the upper and/or lower airways of the person or animal.

In Example 18, the subject matter of any of Examples 1-17 additionally or alternatively includes, removing a majority of the at least one alcohol from the upper and/or lower airways of the person or animal after application of the at least one alcohol to the upper and/or lower airways of the person or animal.

In Example 19, the subject matter of any of Examples 1-18 additionally or alternatively includes, removing fluid and/or mucous from the upper and/or lower airways of the person or animal after application of the at least one alcohol to the upper and/or lower airways of the person or animal.

In Example 20, the subject matter of any of Examples 1-19 additionally or alternatively includes, intubating the person or animal exposed to the infectious agent after or during application of the at least one alcohol to the upper and/or lower airways of the person or animal.

In Example 21, the subject matter of any of Examples 1-20 additionally or alternatively includes, diluting the at least one alcohol prior to application of the at least one alcohol to the upper and/or lower airways of the person or animal.

In Example 22, the subject matter of any of Examples 1-21 additionally or alternatively includes, diluting the at least one alcohol with water and/or saline prior to application of the at least one alcohol to the upper and/or lower airways of the person or animal.

In Example 23, the subject matter of any of Examples 1-22 additionally or alternatively includes, diluting the at least one alcohol with a bronchodilator-containing solution prior to application of the at least one alcohol to the upper and/or lower airways of the person or animal.

In Example 24, the subject matter of any of Examples 1-23 additionally or alternatively includes, diluting the at least one alcohol with surfactant-containing solution prior to application of the at least one alcohol to the upper and/or lower airways of the person or animal.

In Example 25, the subject matter of any of Examples 1-24 additionally or alternatively includes, diluting the at least one alcohol to contain between and including 5% to 95% alcohol or ethanol prior to application of the at least one alcohol to the upper and lower airways of the person or animal.

In Example 26, the subject matter of any of Examples 1-25 additionally or alternatively includes, diluting the at least one alcohol to contain from 30% to 70% alcohol or ethanol prior to application of the at least one alcohol to the upper and/or lower airways of the person or animal.

In Example 27, the subject matter of any of Examples 1-26 additionally or alternatively includes, mixing the at least one alcohol with an at least one additional active pharmaceutical ingredient and/or active pharmaceutical ingredient formulation prior to application of the at least one alcohol to the upper and/or lower airways of the person or animal.

In Example 28, the subject matter of any of Examples 1-27 additionally or alternatively includes, wherein the at least one alcohol includes a liquid pharmaceutical drug formulation having an at least one excipient ingredient and an at least one active pharmaceutical ingredient.

In Example 29, the subject matter of any of Examples 1-28 additionally or alternatively includes, sampling and testing lung fluid and/or lung secretions for infectious agent quantitatively and/or qualitatively prior to and/or after application of the at least one alcohol to the upper and lower airways of the person or animal.

In Example 30, the subject matter of any of Examples 1-29 additionally or alternatively includes, wherein the infectious agent includes a bacteria and/or fungus.

In Example 31, the subject matter of any of Examples 1-30 additionally or alternatively includes, wherein the infectious agent includes a virus and a bacteria.

In Example 32, the subject matter of any of Examples 1-31 additionally or alternatively includes, wherein the infectious agent includes a virus.

In Example 33, the subject matter of Example 32 additionally or alternatively includes, wherein the virus includes a coronavirus.

In Example 34, the subject matter of any of Examples 32-33 additionally or alternatively includes, wherein the virus includes a severe acute respiratory syndrome coronavirus 2 (SARS-CoV-2).

In Example 35, the subject matter of any of Examples 1-34 additionally or alternatively includes, wherein the person or animal is a COVID-19 patient.

In Example 36, the subject matter of any of Examples 1-35 additionally or alternatively includes, wherein the animal is a veterinary patient.

In Example 37, the subject matter of any of Examples 1-36 additionally or alternatively includes, applying at least 0.1 mL of the at least one alcohol to the upper and/or lower airways of the person or animal.

In Example 38, the subject matter of any of Examples 1-37 additionally or alternatively includes, applying at least 1 mL of the at least one alcohol to the upper and/or lower airways of the person or animal.

In Example 39, the subject matter of any of Examples 1-38 additionally or alternatively includes, applying the at least one alcohol to the upper and/or lower airways of the person or animal using positive-pressure or a positive-pressure device.

In Example 40, the subject matter of any of Examples 1-39 additionally or alternatively includes, applying the at least one alcohol to the upper and/or lower airways of the person or animal while using an incentive device and/or inhalation resistance device.

In Example 41, the subject matter of any of Examples 1-40 additionally or alternatively includes, wherein the at least one alcohol as a liquid, solution, or pharmaceutical drug composition comprises at least 10% alcohol or ethanol.

Example 42 is a lung lavage wash solution comprising at least 10% alcohol or ethanol diluted in saline.

Various illustrative components, blocks, configurations, modules, and steps have been described above generally in terms of their functionality. Persons having ordinary skill in the art may implement the described functionality in varying ways for each particular application, but such implementation decisions should not be interpreted as causing a departure from the scope of the present disclosure.

The previous description of the disclosed embodiments is provided to enable a person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as previously described.

What is claimed is:

1. A method, comprising:
applying an at least one alcohol as a liquid, solution, or pharmaceutical drug composition to upper and/or lower airways of a person or animal exposed to an infectious agent.

2. The method of claim 1, wherein the upper and/or lower airways of the person or animal exposed to the infectious agent are disinfected in response to application of the at least one alcohol to the upper and/or lower airways of the person or animal.

3. The method of claim 1, wherein a viral load in the upper and/or lower airways of the person or animal exposed to the infectious agent is reduced in response to application of the at least one alcohol to the upper and/or lower airways of the person or animal.

4. The method of claim 1, further comprising treating the person or animal exposed to the infectious agent via application of the at least one alcohol to the upper and/or lower airways of the person or animal.

5. The method of claim 1, further comprising reducing or eliminating blood clots of the person or animal exposed to the infectious agent in response to application of the at least one alcohol to the upper and/or lower airways of the person or animal.

6. The method of claim 1, further comprising improving respiration of the person or animal exposed to the infectious agent via application of the at least one alcohol to the upper and/or lower airways of the person or animal.

7. The method of claim 1, further comprising improving oxygen saturation of the person or animal exposed to the infectious agent via application of the at least one alcohol to the upper and/or lower airways of the person or animal.

8. The method of claim 1, further comprising preventing a need for mechanical ventilation of the person or animal exposed to the infectious agent via application of the at least one alcohol to the upper and/or lower airways of the person or animal; or reducing a length of time the person or animal exposed to the infectious agent needs mechanical ventilation in response to application of the at least one alcohol to the upper and/or lower airways of the person or animal.

9. The method of claim 1, wherein the at least one alcohol includes ethanol, propanol, isopropyl alcohol, phenol, an alcohol derivative, an alcohol conjugate, or a combination thereof.

10. The method of claim 1, further comprising nebulizing or aerosolizing the at least one alcohol, or humidifying therapeutic gas with the at least one alcohol, for application to the upper and/or lower airways of the person or animal.

11. The method of claim 1, further comprising spraying, dispensing, or dripping the at least one alcohol for application of the at least one alcohol to the upper and/or lower airways of the person or animal.

12. The method of claim 1, further comprising performing an endoscopy, including rhinoscopy, laryngoscopy, bronchoscopy, or a combination thereof, to apply the at least one alcohol to the upper and/or lower airways of the person or animal.

13. The method of claim 1, further comprising performing a lavage, a bronchoalveolar lavage, or a whole lung lavage to apply the at least one alcohol to the upper and/or lower airways of the person or animal.

14. The method of claim 1, further comprising removing a majority of the at least one alcohol from the upper and/or lower airways of the person or animal after application of the at least one alcohol to the upper and/or lower airways of the person or animal.

15. The method of claim 1, further comprising removing fluid and/or mucous from the upper and/or lower airways of the person or animal after application of the at least one alcohol to the upper and/or lower airways of the person or animal.

16. The method of claim 1, further comprising intubating the person or animal exposed to the infectious agent after or during application of the at least one alcohol to the upper and/or lower airways of the person or animal.

17. The method of claim 1, further comprising diluting the at least one alcohol prior to application of the at least one alcohol to the upper and/or lower airways of the person or animal.

18. The method of claim 1, further comprising diluting the at least one alcohol with water and/or saline prior to application of the at least one alcohol to the upper and/or lower airways of the person or animal.

19. The method of claim 1, further comprising diluting the at least one alcohol with a bronchodilator-containing solution, surfactant-containing solution, or a combination thereof, prior to application of the at least one alcohol to the upper and/or lower airways of the person or animal.

20. The method of claim 1, further comprising diluting the at least one alcohol to contain between and including 5% to 95% alcohol or ethanol prior to application of the at least one alcohol to the upper and lower airways of the person or animal.

21. The method of claim 1, further comprising diluting the at least one alcohol to contain from 30% to 70% alcohol or ethanol prior to application of the at least one alcohol to the upper and/or lower airways of the person or animal.

22. The method of claim 1, further comprising mixing the at least one alcohol with an at least one additional active pharmaceutical ingredient and/or active pharmaceutical ingredient formulation prior to application of the at least one alcohol to the upper and/or lower airways of the person or animal.

23. The method of claim 1, wherein the at least one alcohol includes a liquid pharmaceutical drug formulation having an at least one excipient ingredient and an at least one active pharmaceutical ingredient.

24. The method of claim 1, further comprising sampling and testing lung fluid and/or lung secretions for infectious agent quantitatively and/or qualitatively prior to and/or after application of the at least one alcohol to the upper and lower airways of the person or animal.

25. The method of claim 1, wherein the infectious agent includes a bacteria, fungus, virus, coronavirus, severe acute respiratory syndrome coronavirus 2 (SARS-CoV-2), or a combination thereof.

26. The method of claim 1, further comprising applying at least 0.1 mL of the at least one alcohol to the upper and/or lower airways of the person or animal.

27. The method of claim 1, further comprising applying at least 1 mL of the at least one alcohol to the upper and/or lower airways of the person or animal.

28. The method of claim 1, further comprising applying the at least one alcohol to the upper and/or lower airways of the person or animal using positive-pressure, a positive-pressure device, an incentive device, an inhalation resistance device, or a combination thereof.

29. The method of claim 1, wherein the at least one alcohol as a liquid, solution, or pharmaceutical drug composition comprises at least 10% alcohol or ethanol.

30. A lung lavage wash solution comprising at least 10% alcohol or ethanol diluted in saline.
